(19) United States
(12) Patent Application Publication
(10) Pub. No.: US 2012/0157840 A1
(43) Pub. Date: Jun. 21, 2012

(54) USE OF ULTRASOUND IN THE DIAGNOSIS AND TREATMENT OF MULTIPLE SCLEROSIS

(75) Inventor: Paul David Syme, Roxburghshire (GB)

(73) Assignee: Compumedics Ltd., Abbotsford (AU)

(21) Appl. No.: 12/278,149

(22) PCT Filed: Feb. 2, 2007
(86) PCT No.: PCT/IB07/00247

§ 371 Date: Sep. 20, 2010

(30) Foreign Application Priority Data
Feb. 2, 2006 (GB) 0602110.9

(51) Int. Cl. A61B 8/14; A61N 7/00

(52) U.S. Cl. 600/439

(57) ABSTRACT

A method of detecting, diagnosing and treating Multiple Sclerosis in a patient using ultrasound, and in particular transcranial Doppler ultrasonography. The method of diagnosis or detection involves identifying abnormal ultrasound arterial signals, and may also involve establishing one or more characteristic clinical symptoms of Multiple Sclerosis to enable a diagnosis to be made. The method of treatment may involve ultrasound insonation of the cerebral artery.

The present invention relates to the use of ultrasound in diagnosing and treating multiple sclerosis.

Multiple sclerosis (or MS) is a chronic, often disabling auto-immune disease that affects the central nervous system (brain and spinal cord). It is the most common disabling neurological disease among young adults, and affects approximately 85,000 people in the UK. The disorder most commonly begins between the ages of 20 and 40, and is more common in women than in men. An unpredictable disorder, the symptoms of MS vary from person to person and may range from mild to severe and from brief to persistent. The more common symptoms of MS include numbness in the limbs, fatigue and problems with walking, balance and co-ordination. More severe symptoms include total paralysis and loss of vision.

The exact cause of MS is not known, although it is believed to be caused by damage to the myelin sheath, the protective material which surrounds nerve cells, as a result of an abnormal response by the body's immune system. Myelin is a substance rich in lipid which forms layers around the nerve fibres of the brain, optic nerves and spinal cord and acts as insulation for these fibres. This insulation facilitates the transmission of nerve impulses in the CNS. Destruction of the myelin sheath causes nerve impulses to slow down and become blocked, leading to the symptoms of MS.

Destruction of the myelin leaves multiple areas of scar tissue (sclerosis) in the white matter of the CNS. MS lesions or plaques can form in CNS white matter in any location, although they are commonly located in the optic nerves and tracts, through the supratentorial and infratentorial white matter, and along the myelinated tracts of the spinal cord. Typical locations also include the corpus callosum, cerebellar white matter and the corticospinal tracts. Perhaps the most specific lesions in MS are noted in the corpus callosum at the interface with the septum pellucidum.

As symptoms of MS can mimic other neurological disorders, diagnosis is generally made by ruling out other conditions, as well as identifying clinical signs common to MS. A diagnosis of MS may be based on a classical presentation of, for example, optic-neuritis, transverse myelitis, internuclear ophthalmoplegia and paresthesias and on the identification of other neurological abnormalities which may observed through patient history and examination. The patient often presents with one or more of the symptoms described above, for example, numbness or tingling in an extremity, sight abnormalities or difficulties with co-ordination or balance. These symptoms suggest a diagnosis of MS, particularly if the patient has experiences two or more “flare-ups” (or relapses, attacks or exacerbations) interspaced by periods of remission.

Diagnosis is also supported using evidence from ancillary tests, such as cerebrospinal fluid (CSF) examination for oligoclonal banding and MRI. CSF is a clear fluid that circulates in the space surrounding the spinal cord and brain. In an oligoclonal banding test a sample of CSF is taken via a lumber puncture (spinal tap) during which a needle is inserted into the interspace between the vertebra. The CSF sample can then be tested for the presence of oligoclonal bands (immunoglobulins) which suggest inflammation of the central nervous system and may be a sign of multiple sclerosis. This, taken in context of other clinical findings, can assist in the diagnosis of MS.

The most useful and definitive of diagnostic tools in providing a diagnosis of MS is magnetic resonance imaging (MRI). MRI is a branch of nuclear magnetic resonance (NMR), a procedure that involves detecting a molecular spin in a powerful magnetic field. MRI became clinically available in the mid-1980's, and uses powerful magnets and radio waves to construct clear, detailed pictures of tissue. MRI uses powerful magnetic fields that interact with the proton of hydrogen atoms found in the water contained in all body tissues and fluids. A computer then translates the increased energy of the hydrogen nuclei into cross-sectional images. In this manner, MRI scans allow detailed high resolution images of cross-sections of the brain to be generated. Multiple sclerosis lesions typically show up as bright or pale spots on MRI images. Using this, neurologists can not only identify that there have been probable demyelination events, but can also see where those lesions are. Transcranial Doppler ultrasound scanning was invented by Rune Aaslid over 20 years ago. Ultrasound is a medical imaging technique that uses high frequency sound waves to produce images of structures in the body. The technique is based on the pulse-echo principle in which a short burst of ultrasound is emitted from an ultrasound transducer into tissue. When the ultrasound pulse encounters an interface between structures a proportion of the pulse is reflected back towards the transducer. This is often referred to as an echo. These echoes are collected and processed, by timing the period between transmission of the pulse and reception of the echo, in order to generate the image.

Doppler ultrasonography is based on the Doppler Effect first described by the Austrian physicist Christian Andreas Doppler in 1842. This effect depends on the principle that the frequency of sound waves reflected from any object remains the same if the object is stationary, but increases or decreases if the object moves towards or away from the sounds of the source respectively. When the object which is reflected in the ultrasound waves is moving, the frequency of the echoes changes, creating a higher frequency when is moving towards the probe, and a lower frequency when it is moving away from the probe. This frequency shift is called the Doppler frequency shift, and forms the basis of Transcranial Doppler ultrasonograhy (TCD). Its use has been of some assistance in the diagnosis of stroke and the localisation of arterial blockage due to thromboembolism. It has also been used to monitor vasospasm associated with subarachnoid haemorrhage.

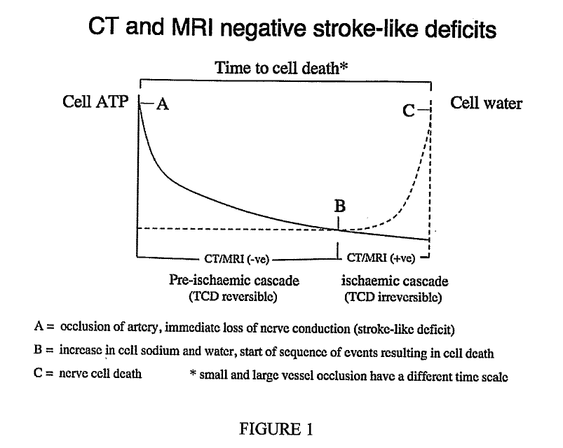

Previous research carried out by the inventor suggests that transcranial Doppler ultrasound scanning can be used to detect small vessel occlusion, and can also be used as a non-invasive method of therapy on its own for small vessel ischemia, including all sub-types of ischemic stroke, ischemia secondary to primary intracerebral haemorrhage and intracerebral tumour. The Applicant's co-pending International Patent Application No WO04/103814 describes new transcranial Doppler ultrasonography findings which have been identified by the inventor as being present in ischemic stroke and small vessel occlusion in general. These high intensity low velocity signals are visible on the ultrasound scan and resemble the short peak systolic wave and diastolic reversal of flow found in circulatory arrest due to brain death. The signal varies from a small triangular noise to a line, although the abnormal signal can also be a bruit and the knock is normally biphasic. Generally the systolic component of the knock can be seen, however a diastolic component is nearly always also observed. The signal repeats with each cardiac cycle, and is generally identifiable at the beginning of systole and at the diacrotic notch. The signals are found in the 300 Hz region of the spectra and have been named “small vessel knock (SVK). As disclosed in WO04/103184 these signals are present in small vessel occlusion and can therefore be used as a diagnostic tool, even in small vessels which are generally too small to allow accurate visualisation in CAT or MRI scans. A proposed mechanism for TCD positivity in small vessel occlusion in relation to MRI negative and positive scans can be seen in FIG. 1. The inventor proposes that following arterial occlusion of small branch arteries or brain perforators the energy substrate in the cell ATP falls to a level that prevents nerve conduction and the patient presents with a stroke-like deficit. With sufficient ATP, Na⁺/K⁺-ATPase the sodium pump of the cell still maintains the water content of the cell and because the water content has not changed the MRI remains negative because there is no increase in cell protons. However, when the ATP falls to a critical stage the water content of the cell rises and the MRI becomes positive. This is a late stage and the cell is close to death. This provides an explanation for stroke-like deficits with normal MRIs and evidence of occlusion from transcranial Doppler ultrasonography. The inventor proposes that the cells remain with impaired conduction but normal water content because of the size of the occlusion and that the neighbouring blood supply is able to keep the ischaemic tissue alive.

Ultrasonography is not currently used in the investigation of MS. The present invention is based on the finding that ultrasound can be used to diagnose Multiple Sclerosis and in some cases also offers a method of treating the symptoms caused by the disorder.

According to a first aspect of the present invention, there is provided the use of ultrasound for the detection of multiple sclerosis in a patient.

According to a second aspect of the present invention there is provided a method of diagnosing multiple sclerosis in a patient by the use of transcranial doppler ultrasonograhy.

Preferably a diagnostic transcranial Doppler ultrasound machine is used. The ultrasound machine will comprise a display for displaying the signal produced in response to ultrasound. Preferably an ultrasound probe of 2 MHz or less is used.

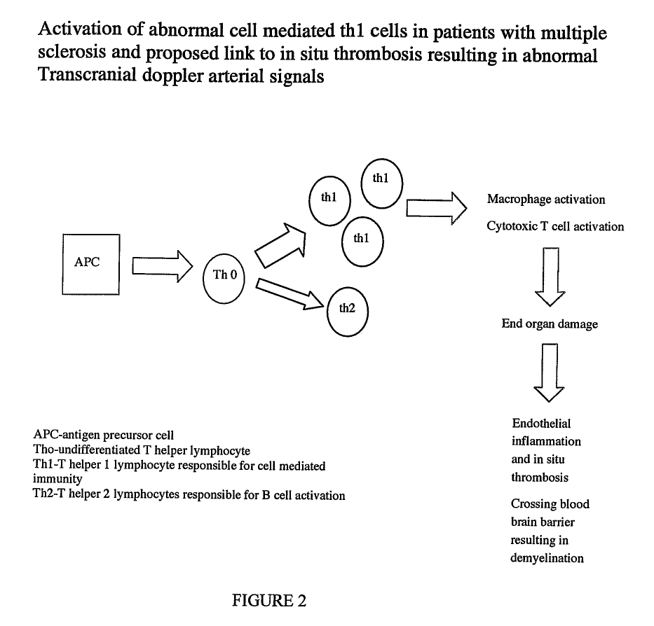

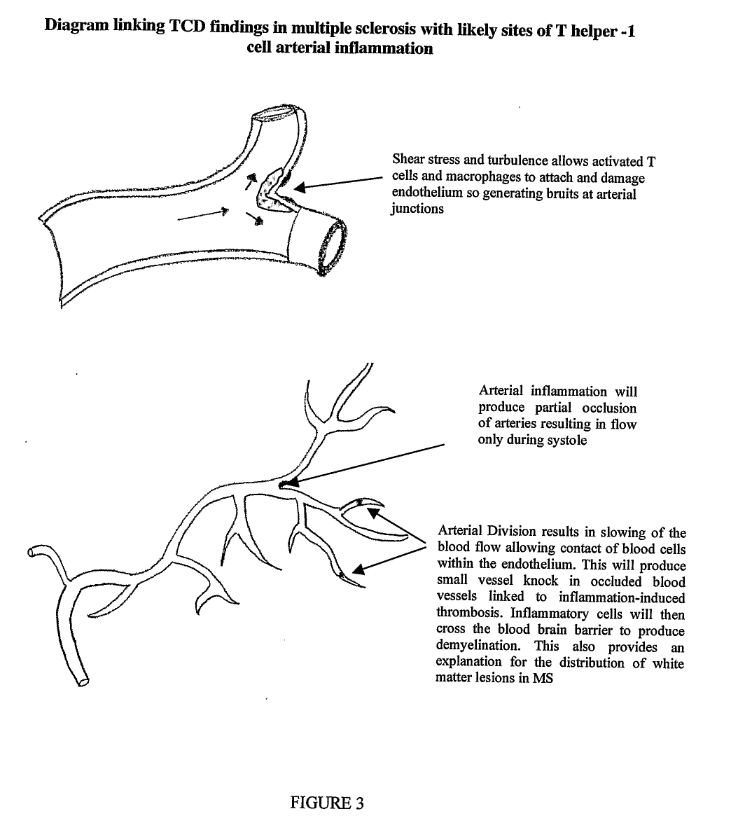

Preferably diagnosis of multiple sclerosis is carried out by the identification of abnormal ultrasound arterial signals. The proposed mechanism for these abnormal signals are illustrated in FIGS. 2 and 3. In FIG. 2 the mechanism which produces abnormal signals in the arteries of MS patients is inflammation of the endothelium and subsequent thrombosis as a result of activation of T helper lymphocytes responsible for cell-mediated immunity (Th1 lymphocytes). The pattern of abnormal artery signals found by Transcranial Doppler ultrasonography is illustrated in FIG. 3. In this arterial damage occurs in sites of turbulence and when the blood flow slows enough to allow adhesion of activated immune cells to the endothelium. This will then set up vascular inflammation and result in bruits at arterial divisions, partial occlusion in smaller arteries and occlusion of smaller arteries including perforators. The diagnosis of multiple sclerosis is usually carried out by the identification of abnormal ultrasound arterial signals in the cerebral artery.

The abnormal ultrasound arterial signals are found at the baseline within the +/−300 Hz range.

Typically the abnormal ultrasound arterial signals are associated with each cardiac cycle and have an intensity which varies according to the rhythm of the patient's heartbeat.

In small vessels the abnormal ultrasound arterial signals typically resemble the short peak systolic wave and diastolic reversal of flow which can be seen with circulatory arrest due to brain death and are high intensity, low velocity signals.

The abnormal ultrasound arterial signals can be seen at the beginning of each systole. The abnormal ultrasound arterial signal may also have a less obvious diastolic component.

Typically the ultrasound arterial signals are biphasic and identifiable at the beginning of systole and at the diacrotic notch.

Typically the ultrasound filter is reduced to 300 Hz or less in order to identify the abnormal ultrasound arterial signals around the baseline.

The presence of the abnormal ultrasound arterial signals, together with one or more characteristic clinical symptoms of the disorder allow a diagnosis of Multiple Sclerosis to be made.

According to a third aspect of the present invention there is provided a method of treating the symptoms of Multiple Sclerosis using ultrasound insonation.

Preferably insonation is carried out using a diagnostic transcranial Doppler ultrasound machine.

Preferably ultrasound insonation is carried out using a 2 MHz probe.

Preferably the method of treatment involves insonation of the cerebral artery, including the anterior cerebral artery.

The method of treatment is preferably carried out following a diagnosis of Multiple Sclerosis made using the method of the first and second aspects.

The method of treatment may also be used in conjunction with MRI screening for Multiple Sclerosis lesions in the brain. Typically the method will be used on patients found to have the abnormal ultrasound arterial signals of the first and second aspects but a negative MRI result.

According to a fourth aspect of the present invention there is provided a method of treating Multiple Sclerosis comprising the steps of:
(a) Making a diagnosis of Multiple Sclerosis using one or more clinical methods such as assessment of symptoms, patient history and oligoclonal band testing of the CSF;
(b) Identifying whether the patient would benefit from ultrasound treatment by screening for the presence of small vessel knock in the cerebral artery using the method of the first and second aspects, and carrying out MRI imaging to determine if plaques are present;
(c) Insonating the cerebral artery, to target the area of occlusion, on high power (e.g., often in the region of 100 Mwatts and above) until signs of clinical improvement are observed.

In the present Application, all reference to research is entirely attributable to Dr Paul Syme, who is the inventor.

The inventor's current research indicates that transcranial Doppler ultrasound can be used to diagnose Multiple Sclerosis and that a link exists between small vessel occlusion and this disorder. This is based on the finding that the signals found on the ultrasound scan when insonating a number of patients with Multiple Sclerosis are identical to those found in elderly patients having small vessel occlusion. These signals take the form of small vessel knock, i.e. are high intensity low velocity signals, which are normally biphasic and normally have a systolic component which repeats with each cardiac cycle. A diastolic component is nearly always also observed. The signals are identifiable at the beginning of systole and at the diacrotic notch and are found in the 300 Hz region of the spectra.

The inventor's research indicates that small vessel knock can be observed in the cerebral artery, and in particular the anterior cerebral artery, of Multiple Sclerosis patients. In particular the inventor's research suggests a link between small vessel knock in the anterior cerebral artery and Multiple Sclerosis.

There are three windows for insonation of the cerebral artery—the first being the temporal area of the skull, known as the “transtemporal window” between the corner of the eye and the pinna of the ear above the zygomatic arch. The second is the transforaminal window at the back of the head which allows insonation of the posterior circulation. The third window is the transorbital window through the eye although insonation through the eye is currently restricted for fear of heating the retina. As a result, power currently needs to be reduced to no more than 17 mW or a tenth of maximum. As a result, current techniques concentrate insonation on the “transtemporal window” as it provides a “window” to the cerebral circulation. This area is preferred for insonation as it is the thinnest area of the skull and has less cancellous bone than other regions. As a result there is less attenuation of the ultrasound by tissue. However one difficulty with insonation in this region is that the ultrasound waves effectively enter the tissue at an angle to the most anterior parts of the anterior cerebral artery and as a result not all of this artery can be studied. Based on his current research the inventor hypothesises that if insonation is directed upwards and towards the back of the head through the cribriform plate of the skull, which divides the top of the nasal cavity from the anterior cranial cavity and the frontal plate, and the superior orbital plate, the most anterior parts of the artery can be seen. Insonation through the eye should allow anterior cerebral artery to be viewed beyond the segment currently visible by transtemporal insonation. This is because the sound has to travel at less than 30 degrees to the artery to see the signal. Beyond the second part of the artery the artery is at 90 degrees to the probe at the transtemporal position. However, from the orbit aiming upwards a probe could be designed to avoid the retina and insonate through the roof of the orbit to see the currently invisible part of the anterior cerebral artery. This is important for the treatment of MS which involves lesions in the corpus callosum which are in distal anterior cerebral artery territory. This would also be the case for vascular dementia which involves the frontal lobe supplied by the anterior cerebral artery. This area is also particularly thin which would facilitate insonation.

Use of this method has indicated that small vessel knock is associated with white matter lesions characteristic of MS. In four patients with Multiple Sclerosis, vascular disease evidenced by the presence of small vessel knock on the ultrasound scan was identified. This has led the inventor to hypothesise that small vessel vasculitis leading to vessel occlusion may be an important factor in the development of Multiple Sclerosis and indeed that demyelination may be a particular form of small vessel occlusion.

Using the methods described herein a new method of diagnosing MS using ultrasound insonation is described. The method involves insonating the cerebral artery, and in particular the anterior cerebral artery using Doppler ultrasound at high power. MS patients are typically found to have lesions all over the brain and most of these are insonatable through the current ultrasound transtemporal and transforaminal windows. However to view the anterior cerebral artery lesions in the distal distribution of this artery would require the use of a new technique to be developed through the roof of the orbit as postulated by the inventor. The presence of “small vessel knock” signals of the type discussed in this Application and the Applicant's co-pending International Application No WO04/103814, together with clinical symptoms characteristic of the disorder suggest a diagnosis of Multiple Sclerosis should be made.

The present invention also provides a method of treating the symptoms of Multiple Sclerosis. The method of the present invention uses a diagnostic Transcranial Doppler ultrasound machine (such as Ezdop DWL Doppler box) and is carried out after clinical diagnosis of MS is established, using a combination of one or more of patient history, oligoclonal banding and ultrasound screening for small vessel knock in the cerebral artery. It would appear that patients with MRI negative lesions (and the presence of small vessel knock) are most likely to improve with ultrasound treatment. The inventor's findings suggest that MRI invisible lesions are particularly sensitive to this technique and show reversibility following insonation treatment and it is believed this is because cell death has not yet occurred. This is illustrated for vessel occlusion in FIG. 1. When a vessel is occluded and blood supply is obstructed the lack of oxygen switches off conduction in neuronal cells stopping the production of ATP, the high energy phosphate molecule required to provide energy for cellular function. As neurones need a large amount of ATP to function efficiently this ischeamia stops conduction. However, the cell integrity and water content of the cells is maintained to the last by the sodium pump, also known as the Na⁺/K⁺-ATPase, which is responsible for establishing and maintaining the electrochemical gradient in animal cells. This enzyme is a component of the plasma membrane and transports Na⁺ and K⁺ using ATP hydrolysis. For every molecule of ATP hydrolyzed, three Na⁺ ions from the intracellular space and two K⁺ ions from the external medium are exchanged. Thus, the sodium pump contributes substantially to the maintenance of the membrane potential of the cell, provides the basis for neuronal communication, and contributes to the osmotic regulation of the cell volume. The sodium pump has a greater affinity for ATP and maintains the cell sodium and water at a constant level thereby preventing cell death until the ATP drops to a critical level. At this point the sodium and calcium build up in the cell and the cell water content changes. This is likely to be the reason why MRI then detects a positive lesion. However, at this MRI visible stage the cell is close to death. It is possible that with opening of the artery some of these areas will survive but they may not. MRI positivity does not suggest a good likelihood of success, and therefore if an MRI positive finding of MR is obtained it is believed it will likely be too late to treat the patient using the method herein described. However using the present method it may be possible to detect small vessel knock linked to early demyelination before the MRI becomes positive, and during which time ultrasound insonation may be effective. Sound targeting could also be used in conjunction with other modealities such as MRI. MRI will show MRI positive lesions but TCD could scan for TCD positive MRI negative lesions and targeted ultrsound could be used to open these vessels. This invention could therefore be used as a screening and treatment tool resulting in the prevention of MRI positive lesions which may be permanent.

Insonation at high power can therefore be used for MRI negative TCD targetable lesions. The power required will vary and depends on the depth of the lesions. For the same power the pulse repetition frequency falls (less packets of ultrasound energy) with increasing depth. This can be overcome by increasing the sample volume and by increasing the power. Power sample volume and the PRF(reflected by the scale) are all connected. In the present methods Doppler ultrasound with a frequency of 2 MHz and diameter of 1.6 cm is used. The power for M mode is expressed in mW/cm2. The power required will vary, and for other frequencies will depend on the diameter of the probe head. The power from the probe depends on the pulse repitition frequency (PRF) which also depends on the sample volume and the depth of insonation. For any power the deeper the signal the lower the power should be used. For the method of treatment herein described the power should be maximum but kept for as short a time as possible. For diagnostic insonation the power should be kept as low as possible. It follows that for safety reasons, the technique should use as low power as possible to identify single-gated SVK and M-mode SVK. When the signals are identified the parameters should be adjusted to maximise the power.

In addition the duration of insonation should be kept as short as possible. In particular the thermal cranial index (TIC=Wₜ/40*d) which is a standard thermal index derived from estimating maximum heating of tissues while accounting for signal attenuation due to cranial bone should be observed, and should always be kept below a value of 2.0. One or more of the parameters of intensity, sample volume or scale, should therefore be adjusted during insonation to maintain the TIC at or below 2.0. If a TIC value of more than 2.0 cannot be avoided the insonation time should be reduced to 15 minutes. At greater depths the duration of insonation will be greater when targeting deeper SVK signals than for more superficial SVK signals.

Insonation at a power of, for example, 200 MWatts can be used although lower power could also be used with some success.

The therapeutic method of the present invention can be carried out as follows:
(a) Identification of Multiple Sclerosis using clinical methods such as assessment of symptoms, patient history and oligoclonal band testing of the CSF.
(b) Identification of whether the patient would benefit from ultrasound treatment by screening for the presence of small vessel knock in the cerebral artery, and MRI imaging to determine if plaques are present. A finding of small vessel knock with MS symptoms but normal MRI/MRI invisibility suggests patient would benefit from ultrasound treatment and recovery is possible.
(c) Insonation of the cerebral artery, to target the area of occlusion, on high power (e.g., often in the region of 100 Mwatts and above) until signs of clinical improvement are observed.

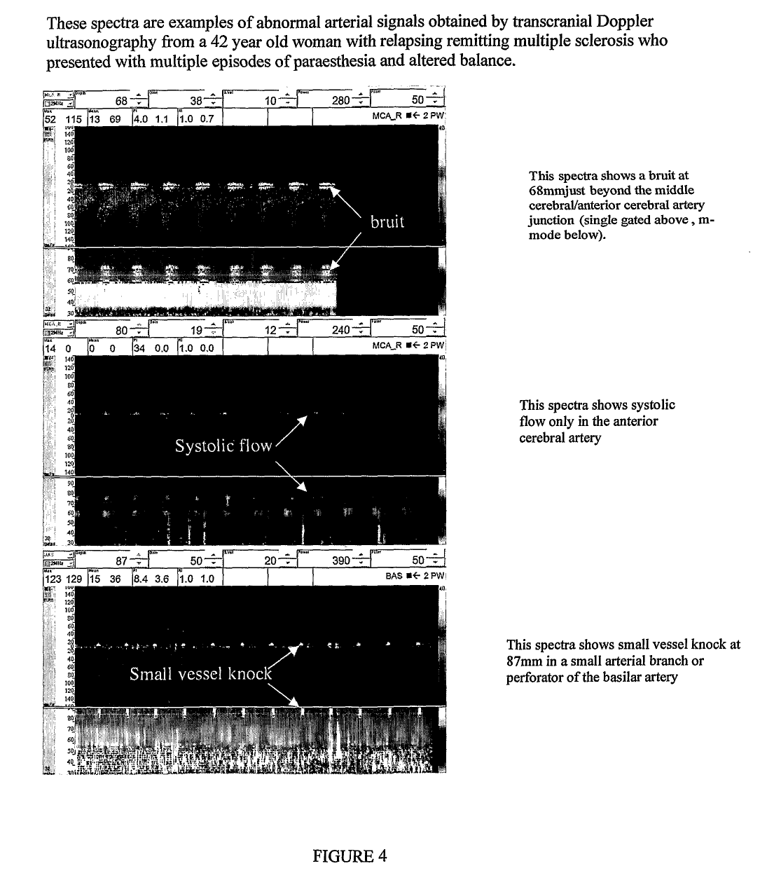

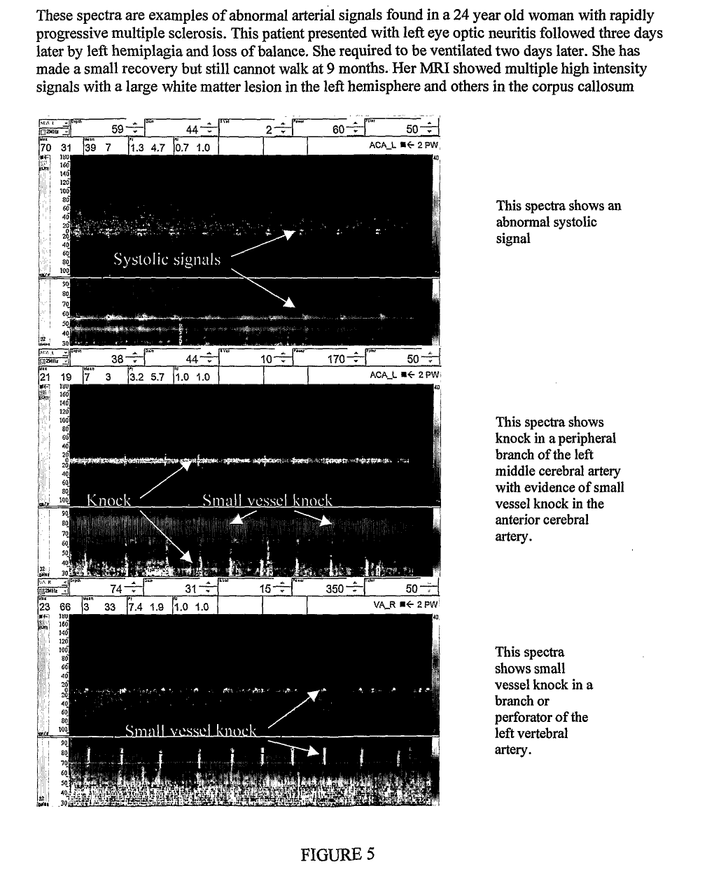

To serve purely as illustrations of the signals detected by Transcranial Doppler ultrasonography FIGS. 4 and 5 show examples of arterial signals detected in two proven cases of MS.

It should be noted that the embodiments disclosed above are merely exemplary of the invention, which may be embodied in different forms. Therefore details disclosed herein are not to be interpreted as limiting, but merely as a basis for claims and for teaching one skilled in the art as to the various uses of the present invention in any appropriate manner.

1. A method of detecting or diagnosing Multiple Sclerosis in a patient, comprising the steps of: using ultrasound to detect or diagnose Multiple Sclerosis in a patient.

2. A method as claimed in claim 1, wherein Multiple Sclerosis is diagnosed by the use of transcranial Doppler ultrasonography.

3. A method as claimed in claim 2, wherein a diagnostic transcranial Doppler ultrasound machine is used.

4. A method as claimed in claim 3, wherein the ultrasound machine comprises a display for displaying the signal produced in response to ultrasound.

5. A method as claimed in claim 2, wherein an ultrasound probe of 2 MHz or less is used.

6. A method as claimed in claim 2, wherein the method further comprises the identification of abnormal ultrasound arterial signals.

7. A method as claimed in claim 6, wherein the method further comprises the identification of abnormal ultrasound arterial signals in the cerebral artery.

8. A method as claimed in claim 6, wherein the abnormal ultrasound arterial signals are found at the baseline within the +/−300 Hz range.

9. A method as claimed in claim 6, wherein the abnormal ultrasound arterial signals are associated with each cardiac cycle.

10. A method as claimed in claim 6, wherein the abnormal ultrasound arterial signals have an intensity which varies according to the rhythm of the patient's heartbeat.

11. A method as claimed in claim 6, wherein the abnormal ultrasound arterial signals typically resemble the short peak systolic wave and diastolic reversal of flow which can be seen with circulatory arrest due to brain death.

12. A method as claimed in claim 6, wherein the abnormal ultrasound arterial signals are high intensity, low velocity signals.

13. A method as claimed in claim 6, wherein the abnormal ultrasound arterial signals are identified at the beginning of each systole.

14. A method as claimed in claim 13, wherein the abnormal ultrasound arterial signal comprises a diastolic component.

15. A method as claimed in claim 6, wherein the abnormal ultrasound arterial signals are biphasic and identifiable at the beginning of systole and at the diacrotic notch.

16. A method as claimed in claim 6, wherein the ultrasound power is reduced to 2 MHz or less in order to identify the abnormal ultrasound arterial signals.

17. A method as claimed in claim 6, wherein the presence of the abnormal ultrasound arterial signals, together with one or more characteristic clinical symptoms of Multiple Sclerosis allows a diagnosis to be made.

18. A method of treating the symptoms of Multiple Sclerosis, the method comprising the steps of: using ultrasound insonation to treat the symptoms of Multiple Sclerosis.

19. A method as claimed in claim 18, wherein insonation is carried out using a diagnostic transcranial Doppler ultrasound machine.

20. A method as claimed in claim 18, wherein ultrasound insonation is carried out using a 2 MHz probe.

21. A method as claimed in claims 18, wherein the method of treatment involves ultrasound insonation of the cerebral artery.

22. A method as claimed in claim 21, wherein the method of treatment includes ultrasound insonation of the anterior cerebral artery.

23. A method as claimed in claim 18, wherein the method of treatment is carried out following the method of diagnosis described in claims 1 to 17.

24. A method as claimed in claim 18, wherein the method of treatment is used in conjunction with MRI screening for Multiple Sclerosis lesions in the brain.

25. A method as claimed in claim 18, wherein the method of treatment is used on patients found to have abnormal ultrasound arterial signals but a negative MRI result.

26. A method of treating Multiple Sclerosis in a patient comprising the steps of:
(a) making a diagnosis of Multiple Sclerosis using one or more clinical methods;
(b) identifying whether the patient would benefit from ultrasound treatment by screening for the presence of small vessel knock in the cerebral artery using the method as described in claims 1 to 17, and carrying out MRI imaging to determine if plaques are present; and
(c) identifying the cerebral artery, to target the area of occlusion until signs of clinical improvement are observed.

27. A method as claimed in claim 26, wherein the clinical methods of diagnosis comprise one or more of: assessment of symptoms, patient history and oligoclonal band testing of the CSF.

28. A method as claimed in claim 26, wherein the area of occlusion is targeted with ultrasound in the region of 100 Mwatts and above.
